(19) United States
(12) Patent Application Publication
(10) Pub. No.: US 2024/0371465 A1
(43) Pub. Date: Nov. 7, 2024

(54) METHOD, SYSTEM, AND COMPUTER READABLE MEDIUM FOR POST-TRANSLATIONAL MODIFICATIONS DETECTION

(71) Applicant: National Central University, Taoyuan City (TW)

(72) Inventors: Hui-Yin Chang, Nantou County (TW); Yi-Fan Chen, Hsinchu City (TW); Jie-Wei Chiu, Taichung City (TW)

(73) Assignee: National Central University, Taoyuan City (TW)

(21) Appl. No.: 18/142,035

(22) Filed: May 2, 2023

(51) Int. Cl. G16B 20/20; G16B 15/00; G16B 30/10

(52) U.S. Cl. CPC G16B 20/20; G16B 15/00; G16B 30/10

(57) ABSTRACT

Provided are a method and a system for automatic detection of post-translational modifications (PTMs) detection, where a depth-first search (DFS) algorithm is utilized to generate all post-translational modification combinations and respective masses thereof based on all PTMs associated with compound-spectrum matches of a biological compound, such that a mass shift of a compound-spectrum match of the biological compound generated by search engines with the open search strategy can then be processed by the system and the method provided herein. Therefore, users can validate the search results from the open search via the same search engines with closed strategy for precise identification and discovery of potential unanticipated modifications of the biological compound. Also provided is a computer readable medium with executable instructions stored thereon to perform the method of the present disclosure.

### BACKGROUND OF THE INVENTION

### 1. Field of the Invention

The present disclosure relates to a method, system, and computer readable medium for database searching, and more particularly to a soft tool or platform for detection of post-translational modifications of a biological compound.

### 2. Description of the Prior Art

In the field of bottom-up proteomics (often referred as shotgun proteomics), implying protein database search for peptide identification and quantification is a conventional technique to identify and quantify thousands of proteins and their post-translational modifications (PTMs) in complex biological mixtures. In particular, identification of peptide and their PTMs via high performance liquid chromatography coupled with tandem mass spectrometry (HPLC-MS/MS), which the proteins extracted from cells or tissues are enzymatically digested to peptides then separated by liquid chromatography and analyzed by mass spectrometer, may reflect the functionality and diversity of the protein. The acquired tandem mass spectra (MS/MS) are matched to peptide candidates through calculating the correlation between in silico spectra with those MS/MS spectra, resulting numerous peptide-spectrum matches (PSMs).

Existing database search tools supporting closed search strategy (also called restrictive protein sequence database search) for identifying the above PSMs have been proposed, such as SEQUEST, MS Amanda, and MSFragger, which precisely provide the identification of the peptide sequences using a tight peptide precursor mass tolerance (e.g., 20 ppm). However, prior knowledge of PTMs potentially exist in a sample when performing database searches has to be specified, because the search space is mainly composed by protein sequences and PTMs (An, Z., et al. Mol Cell Proteomics 2019, 18 (2), 391-405). Furthermore, the setting of modification often depends on the basis of experimental design (e.g., chemical mass tags used in a labeling proteomic experiment need to be included as the fixed modification for closed searches), or users' experiences. That is to say, these tools highly depend on the correct specification of modifications and most search engines only allow a limited number of modifications. Peptides with unspecified or user-unknown modifications are therefore lost, resulting numerous unexplained MS/MS spectra (Chick, J. M., et al. Nat Biotechnol 2015, 33 (7), 743-749).

An alternative solution, open search (or called mass-tolerant search), has been proposed to address this issue which applies a much wider precursor mass tolerance (e.g., 500 Da.) and requires less prior knowledge of modification settings. Its major advantage is the ability of identifying a large proportion of unassigned MS/MS spectra as modified peptides. Despite its high flexibility, the open search strategy has not been widely applied in current proteomic research due to two major challenges. The first challenge is the dramatically increased search space resulted by the enlarged precursor mass tolerance. Several search engines have been developed to address this issue, such as MSFragger, Open-pFind, MODa, MetaMorpheus, and TagGraph. The second challenge is the unknown mass shifts between the observed precursor and peptide candidates, which possibly correspond to noises, sequence variants, or potential undiscovered modifications. Some software tools with sophisticated algorithms, such as PTM-shepherd, PTMiner, and AA_stat, has been recently proposed to profile and annotate PTMs from the unknown mass shifts between the observed and theoretical precursor masses.

Based on the above, there is an unmet need in the art to solve the aforementioned problems and utilize both the advantages of the closed and open search strategies in the proteomics, i.e., accurate peptide sequence identified by closed searches and potential unanticipated modification discovered by open searches, for identifying peptides and their modifications. Although several tools have been proposed for detecting PTMs from the open search results, most of them neglect the fact that a mass shift possibly corresponds to a combination of PTMs, in addition to a single PTM. Therefore, a variable-modification-setting-free approach as a bridge to connect closed and open search for uncovering potential protein PTMs are of high value and still necessary in the art.

### SUMMARY OF THE INVENTION

In view of the foregoing, the present disclosure provides a method for automatic detection of post-translational modifications of a biological compound, and the method comprising: acquiring a mass shift of a compound-spectrum match between the biological compound and a spectrum; generating post-translational modification combinations and masses thereof by processing user-defined post-translational modifications using a search algorithm; matching the mass shift and a mass of each of the post-translational modification combinations; and automatically detecting the post-translational modifications of the biological compound based on the matching.

The present disclosure further provides a system for automatic detection of post-translational modifications of a biological compound, and the system comprises a memory and a processor. The memory is used for storing a user-defined post-translational modifications and a mass shift of a compound-spectrum match between the biological compound and a spectrum. The processor is used for generating post-translational modification combinations and masses thereof by processing the user-defined post-translational modifications using a search algorithm; matching the mass shift and a mass of each of the post-translational modification combinations; and automatically detecting the post-translational modification combinations of the biological compound based on the matching.

In at least one embodiment of the present disclosure, the method further comprises using a position obtained from the compound-spectrum match to validate the correctness of matched post-translational modification combinations. In some embodiments, the post-translational modification combinations of the biological compound is detected by the following conditions: 1) a mass difference between the mass shift of the compound-spectrum match and the mass of each of the post-translational modification combinations is within a default value, and 2) locations of post-translational modifications in the post-translational modification combinations are included in a sequence of the compound-spectrum match. In some embodiments, the method further comprises exporting matched post-translational modification combinations mapping to the compound-spectrum match. In at least one embodiment of the present disclosure, the processor is used for executing the method mentioned above.

In at least one embodiment of the present disclosure, the search algorithm is a depth-first search algorithm. In some embodiments, each of the user-defined post-translational modifications is a tree node of the depth-first search. In some embodiments, the number of the user-defined post-translational modifications may be 100 or more.

In at least one embodiment of the present disclosure, the biological compound is a protein. In some embodiments, the compound-spectrum match is a peptide-spectrum match (PSM).

In at least one embodiment of the present disclosure, the mass shift is a difference between a theoretical mass and a measured mass of a compound-spectrum match.

In at least one embodiment of the present disclosure, each of the post-translational modifications contains up to N post-translational modifications, and N is a number defined by users. In some embodiment, each of the post-translational modifications contains up to 3 or 4 post-translational modifications. In some embodiment, each of the post-translational modifications contains 1, 2, or 3 post-translational modifications (i.e., default value of N is 3).

In at least one embodiment of the present disclosure, the method further comprises acquiring the compound-spectrum match and a mass thereof from an open search strategy. In some embodiments, the number of compound-spectrum matches in the open search result is over fifty thousand. In at least one embodiment of the present disclosure, the processor is used for executing the method mentioned above.

In at least one embodiment of the present disclosure, the method further comprises exporting the post-translational modification combinations to a closed search strategy for identification and validation. In some embodiments, an identified result of the closed search strategy is validated by a validation tool such as Percolator, PeptideProphet, or ProteinProphet, but the present disclosure is not limited thereto. In at least one embodiment of the present disclosure, the processor is used for executing the method mentioned above.

In at least one embodiment of the present disclosure, the method combines the open search strategy and the closed search strategy mentioned above to automatically detect post-translational modifications of the biological compound. In at least one embodiment of the present disclosure, the processor is used for executing the method mentioned above.

The present disclosure also provides a computer readable medium with computer executable instructions stored thereon to perform the method described above.

The present disclosure can automatically detect PTMs from the biological compound data (e.g., proteomics data), and more information thereof such as PSMs, peptides, and proteins can be identified using modifications recommended by the preset disclosure. Moreover, by using a search algorithm such as a depth-first search algorithm, the present disclosure can efficiently generate millions of post-translational modification combinations and masses thereof on the basis of user-defined post-translational modifications. As such, unexpected PTMs can be discovered from the biological compound data via the present disclosure that maps the masses of the post-translational modification combinations and the mass shifts. In addition, the present application also show excellent flexibility and robustness when used with different search engines (e.g., MSFragger and Metamorpheus), and the performance thereof is stable and outperformed. Therefore, the present disclosure provides an approach for users to utilize both the advantages of the closed and open search strategies that can effectively validate the search results from the open search via the same search engines with the closed strategy for precise identification and discovery of potential unanticipated modifications of the biological compounds.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The present disclosure will become more readily appreciated by reference to the following descriptions in conjunction with the accompanying drawings.

FIG. 1 is a flow chart illustrating steps for protein post-translational modifications detection in accordance with the present disclosure.

FIGS. 2A and 2B show the overview of protein PTMs identification workflow in accordance with the present disclosure. FIG. 2A is a flow chart illustrating steps for the general workflow of AutoMod, and FIG. 2B is a schematic diagram illustrating the concept of AutoMod algorithm in accordance with the present disclosure.

FIG. 3 is a schematic diagram illustrating an exemplifying structure of a system for automatic detection of post-translational modifications of a biological compound in accordance with the present disclosure.

FIGS. 4A, 4B, and 4C are bar graphs illustrating the number of identified PSMs, peptides with modifications, and proteins using the four different closed search parameter settings, respectively. The white bars represent the number of PSMs, peptides, and proteins commonly identified by the four search parameter settings. The bars with backslashes represent the PSMs, peptides, and proteins identified by the parameter settings of default, customized, and all. The bars with slashes represent the PSMs, peptides, and proteins additionally identified by the parameter settings of default, customized, and all.

FIGS. 5A, 5B, and 5C are Venn diagrams illustrating the number of PSMs, peptides and proteins, respectively, identified using the closed searches with no variable modification, default variable modifications, and AutoMod-recommended modifications. FIGS. 5D, 5E, and 5F are bar graphs showing the peptideprophet probability, peptide probability and protein probability of PSMs, respectively, identified using the default, and AutoMod-recommended searches. The symbol of “=”, “>”, “<” indicate the probability of PSMs, peptides, and proteins identified using the AutoMod-recommended search are equal, larger and smaller than those identified using the default search, respectively.

FIG. 6 is a bar graph illustrating the number of modifications identified using the default and AutoMod search.

FIGS. 7A, 7B, and 7C are bar graph illustrating the number of PSMs, peptide and proteins, respectively, identified using the default and AutoMod search via Metamorpheus.

### DETAILED DESCRIPTION

The following embodiments are provided to illustrate the present disclosure in detail. A person having ordinary skill in the art can easily understand the advantages and effects of the present disclosure after reading this disclosure, and also can implement or apply in other different embodiments. Therefore, any element or method within the scope of the present disclosure disclosed herein can combine with any other element or method disclosed in any embodiment of the present disclosure.

The proportional probabilities, positions, numbers and other features shown in accompanying drawings of this disclosure are only used to illustrate embodiments described herein, such that those with ordinary skill in the art can read and understand the present disclosure therefrom, of which are not intended to limit the scope of this disclosure. Any changes, modifications, or adjustments of said features, without affecting the designed purposes and effects of the present disclosure, should all fall within the scope of technical content of this disclosure.

Unless otherwise specified, wordings in singular forms such as “a,” “an” and “the” also pertain to plural forms, and wordings such as “or” and “and/or” may be used interchangeably.

As used herein, the terms “comprise,” “comprising,” “include,” “including,” “have,” “having,” “contain,” “containing,” or any other variations thereof are intended to cover a non-exclusive inclusion. For example, a combination, tool, process or method that comprises a list of elements is not necessarily limited to only those elements, but may include other elements not expressly listed, or inherent to such composition, combination, process, step or method.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified.

As used herein, the terms “one or more” and “at least one” may have the same meaning and include one, two, three, or more.

The numeral ranges used herein are inclusive and combinable, and any numeral value that falls within the numeral scope herein could be taken as a maximum or minimum value to derive the sub-ranges therefrom. For example, it should be understood that the numeral range “−150 Da to 2000 Da” comprises any sub-ranges between the minimum value of −150 Da to the maximum value of 2000 Da, such as the sub-ranges from −150 Da to 350 Da, from 350 Da to 850 Da, from 850 Da to 1350 Da, from 1350 Da to 1850 Da, from 1850 Da to 2000 Da and so on. In addition, a plurality of numeral values used herein can be optionally selected as maximum and minimum values to derive numerical ranges. For instance, the numerical ranges of −150 Da to 1000 Da, −150 Da to 2000 Da, and 1000 Da to 2000 Da can be derived from the numeral values of −150 Da, 1000 Da, and 2000 Da.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making combinations, matches, validations, calculations or correlations, the differences in the source, tools, or platforms of calculation, searching and matching used in the present disclosure, or like considerations. Alternatively, the term “about” means the numerical value within an acceptable standard error of the mean for a person of ordinary skill in the art. Unless otherwise expressly specified, all of the numerical ranges, numbers, values and probabilities such as those for calculations of matches, combinations, degree of confidence, operating algorithms, tolerances, and the likes disclosed herein should be understood as modified in all instances by the term “about.”

As used herein, the term “biological compound” refers to a compound or molecule that is of biological origin. In at least one embodiment of the preset disclosure, the biological compound may be peptides, proteins, carbohydrates, lipids, glycoproteins, lipoproteins, phosphoproteins, or metabolites. In some embodiments, the biological compound may be proteins. In some embodiments, the biological compound may be methylation variants of the proteins, acetylated or phosphorylated variants of the proteins, ubiquitylation variants of the proteins, extracellular or intracellular proteins, hydroxylation variants of the proteins, sulfation variants of proteins, adducts of metabolites, or metabolite fragments, but the present disclosure is not limited thereto.

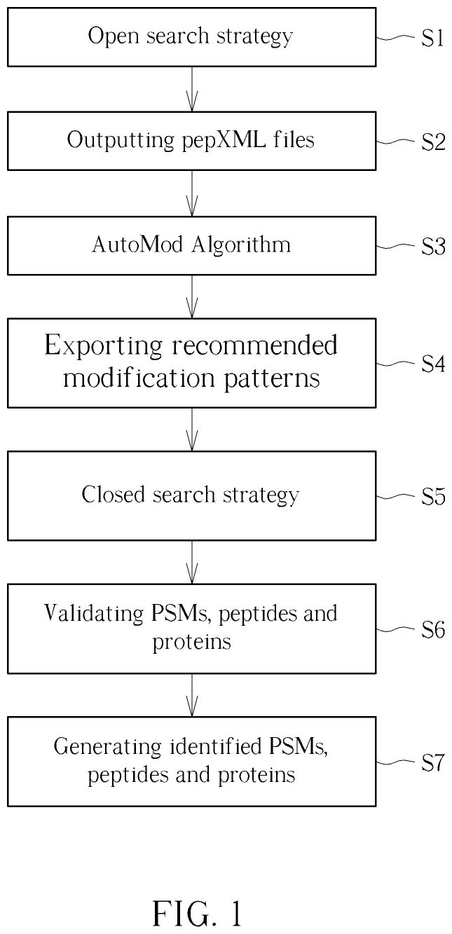

Referring to FIG. 1, a flow chart describing steps for detection of a protein PTM is illustrated, comprising the operation relationships between said elements of the method, which are denoted as arrows (described as “step(s)” herein) and explained herefrom.

In some embodiments, step S1 denotes that the input files in raw or mzML file format are firstly searched against protein databases using the open search strategy, generating numerous pepXML files of PSMs in step S2. For example, the open search strategy may be based on any one of database search engines, software tools, or search algorithms, including MSFragger (Kong, A. T., et al. Nat Methods 2017, 14 (5), 513-520), MetaMorpheus (Solntsev, S. K., et al. J. Proteome Res 2018, 17 (5), 1844-1851), PTM-shepherd (Geiszler, D. J., et al. Mol Cell Proteomics 2021, 20, 100018), PTMiner (An, Z., et al. Mol Cell Proteomics 2019, 18 (2), 391-405), or the like or any combination of those mentioned above, of which the present disclosure is not limited thereto.

In some embodiments, the precursor mass tolerance of open search strategy is between −150 to 2000 Da, such as −150 to −50 Da, −50 to 50 Da, 50 to 150 Da, 150 to 250 Da, 250 to 350 Da, 350 to 450 Da, 450 to 550 Da, 550 to 650 Da, 650 to 750 Da, 750 to 850 Da, 850 to 950 Da, 950 to 1050 Da, 1050 to 1150 Da, 1150 to 1250 Da, 1250 to 1350 Da, 1350 to 1450 Da, 1450 to 1550 Da, 1550 to 1650 Da, 1650 to 1750 Da, 1750 to 1850 Da, 1850 to 1950 Da, 1950 to 2000 Da. In some embodiments, the precursor mass tolerance of open search strategy is about −150 Da, −50 Da, 50 Da, 150 Da, 250 Da, 350 Da, 450 Da, 550 Da, 650 Da, 750 Da, 850 Da, 950 Da, 1050 Da, 1150 Da, 1250 Da, 1350 Da, 1450 Da, 1550 Da, 1650 Da, 1750 Da, 1850 Da, 1950 Da, 2000 Da.

In some embodiments, step S3 denotes that the output pepXML files are then analyzed by AutoMod for the discovery of modification patterns, and exporting recommended modification candidates or patterns in step S4.

In some embodiments, step S5 denotes that the exported modification candidates or patterns are used for closed search strategy. For example, the closed search strategy may be based on any one of database search engines, software tools, or search algorithms, including SEQUEST, Mascot, MS Amanda, MSFragger or the like or any combination of those mentioned above, of which the present disclosure is not limited thereto. Most of the search tools provide two types of PTM settings, including fixed and variable modifications. The fixed (or called static) modification means that the desired PTMs should be applied universally to the assigned amino acid, while the variable (or called dynamic) modification means that the desired PTMs may or may not present in samples (i.e., allowing the absence of PTMs in the assigned amino acids).

In some embodiments, the precursor mass tolerance of closed search strategy is between 1 to 60 ppm, such as 1 to 5 ppm, 5 to 10 ppm, 10 to 15 ppm, 15 to 20 ppm, 20 to 25 ppm, 25 to 30 ppm, 30 to 35 ppm, 35 to 40 ppm, 40 to 45 ppm, 45 to 50 ppm, 50 to 55 ppm, 55 to 60 ppm. In some embodiments, the precursor mass tolerance of closed search strategy is about 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm.

In some embodiments, step S6 denotes that PSMs are validated by conventional validation tools that enable users to perform the conventional identification pipeline with their familiar tools, and therefore exporting high confident PSMs, peptides and proteins in step S7. For example, the conventional validation tools may be based on any one of database search engines, software tools, or search algorithms, including Percolator, PeptideProphet, ProteinProphet or the like or any combination of those mentioned above, of which the present disclosure is not limited thereto.

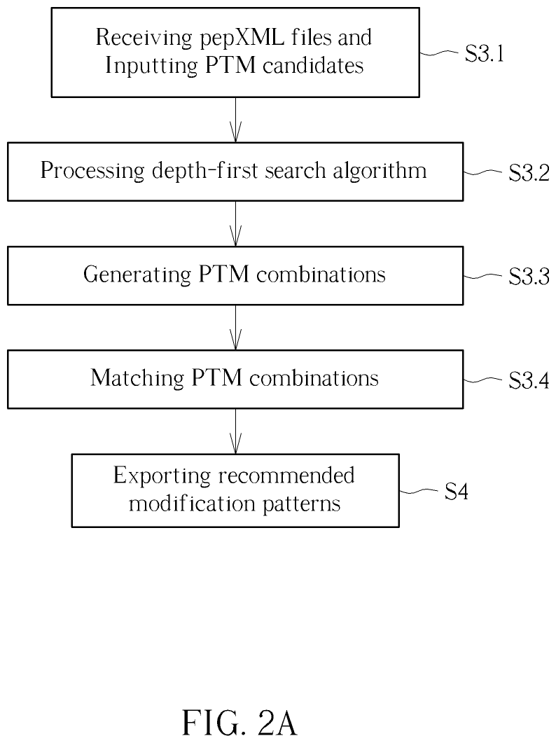

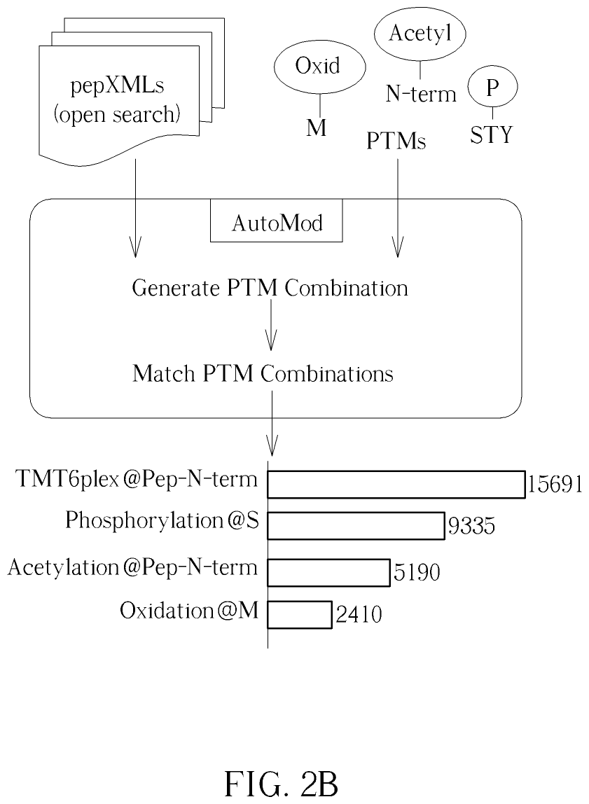

Referring to FIG. 2A, a flow chart describing steps for protein PTMs detection utilizing step S3 of the FIG. 1 is disclosed, where FIGS. 2B and 3-6 are also cited to illustrate execution details for each step by reference.

In some embodiments, step S3.1 denotes that AutoMod takes the pepXML files from open searches and user-defined PTMs as input data.

In some embodiments, steps S3.2 to S3.3 denote that AutoMod first generates the PTM combinations, each with up to N PTMs, where N is defined by users (default value of 3), and computes the sum mass of each PTM combination, using the depth-first search (DFS) algorithm, where each PTM is considered as a tree node.

In some embodiments, step S3.4 denotes that AutoMod applies the mass shift between theoretical and observed precursor masses, for each PSM and possible modified site to match with the generated PTM combinations. If the mass differences between the delta mass and the sum masses of PTM combinations are within mass tolerance (default value of 20 ppm) and the possibly modified sites are included in those of the PTM combinations, the PSM is considered to have the PTM combinations. A PSM might match with more than one PTM combination. AutoMod groups the matched PTM combinations and exports top N PTM candidates, i.e., the PTM matched by most PSMs defined by the users and 10 as the default value.

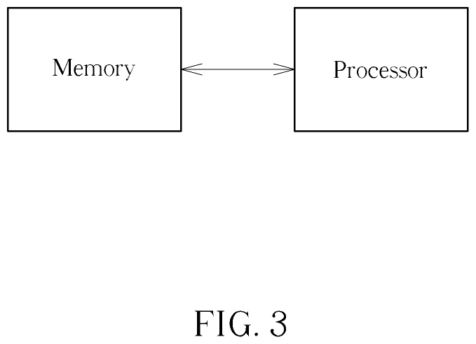

Referring to FIG. 3, a system for automatic detection of post-translational modifications of a biological compound in accordance to the present disclosure is illustrated, comprising: a memory and a processor. Described elements of the system may be connected to each other via any suitable wired or wireless means, of which the present disclosure is not limited thereto. In an embodiment, the memory is configured for storing user-defined post-translational modifications and a mass shift of a compound-spectrum match between the biological compound and a spectrum. The processor is configured for generating post-translational modification combinations and masses thereof by processing the user-defined post-translational modifications using a search algorithm; matching the mass shift and a mass of each of the post-translational modification combinations; and automatically detecting the post-translational modification combinations of the biological compound based on the matching. The Memory in a computer system may include volatile memory and/or non-volatile memory. More specifically, memory may include: ROM, RAM, EPROM, EEPROM, flash, one or more smart cards, one or more magnetic disc storage devices, and/or one or more optical storage devices.

In some embodiments, a computer readable medium is also present, which has computer executable instructions stored thereon, when the computer executable instructions are executed by a processor to cause the processor to perform the steps as discussed in this disclosure.

From here, a detailed description of the general workflow of proteins detection and AutoMod algorithm will be provided.

### Methodology

### Study Datasets

To evaluate the performance of AutoMod, three public datasets were used, including a clear cell renal cell carcinoma (ccRCC) phosphoproteome dataset (Clark, D. J., et al. Cell 2019, 179 (4), 964-983 e931), a HEK293 proteome dataset (Chick, J. M., et al. Nat Biotechnol 2015, 33 (7), 743-749), and an acetylation-enriched proteome dataset (Zhang, L., et al. Mol Cell Proteomics 2022, 21 (9), 100248). The following description of each dataset processing is provided.

### Processing of Study Datasets for Open and Closed Search Strategies

### Clear Cell Renal Cell Carcinoma (ccRCC) Phosphoproteome Dataset

The ccRCC phosphoproteome datasets were downloaded from the CPTAC Data Portal (https://cptac-data-portal.georgetown.edu/study-summary/S044). A total of 110 ccRCC tumor samples, 84 paired normal adjacent tissue (NAT) samples, eight quality control (QC), and five NCI-7 aliquots were randomly assigned to the 23 TMT 10-plex (23 Tandem Mass Tag 10-plex) sets, each having 13 fractions.

The open and closed searches were performed. For the open search, the 299 raw files were converted to mzML file format and searched against a human protein sequence database downloaded from UniProt (retrieved 2022 Apr. 13) using MSFragger. Decoy protein sequences (i.e. fake sequences or unreal sequences) and contaminant protein sequences (i.e. sequences containing one or more sequence segments of foreign origin) were added by Philosopher (da Veiga Leprevost, F., et al. Nat Methods 2020, 17 (9), 869-870). In MSFragger, peptide lower and upper masses of −150 Da. and 500 Da., and fragment tolerance of 20 ppm were used. No variable and fixed modification was applied. Default values were used for the unmentioned parameters. For the closed search, all the mzML files were searched against the same human protein database using MSFragger. Both peptide lower mass and upper mass were set as 20 ppm. Cysteine carbamidomethylation (+57.0215) and lysine TMT labeling (+229.1629) were specified as a fixed modification. In both searches, PeptideProphet (Keller, A., et al. Anal Chem 2002, 74 (20), 5383-5392) and ProteinProphet (Nesvizhskii, A. I., et al. Anal Chem 2003, 75 (17), 4646-4658) were used for peptide and protein validation. The peptide and protein false discovery rate cut-off were set to 0.01.

### HEK293 Proteome Dataset

The label-free HEK293 proteome dataset with 24 fractions was downloaded from ProteomeXchange (PXD001468). For the open search, the 24 raw files were converted to mzML file format and searched against the human protein database using MSFragger. Peptide lower and upper masses of −150 Da. and 500 Da., and fragment tolerance of 20 ppm were used. No variable and fixed modification was applied. Default values were used for the unmentioned parameters. For the closed search, all mzML files were searched against the same human protein database using MSFragger. Peptide lower mass and upper mass were set as 20 ppm. Cysteine carbamidomethylation (+57.0215) and lysine TMT labeling (+229.1629) were specified as a fixed modification. In both of the searches, the peptide and protein false discovery rate cut-off were set to 0.01.

### Acetylation-enriched Proteome Dataset

The label-free acetylation-enriched proteome dataset with 3 replicates was downloaded from ProteomeXchange (PXD028447). The raw files were converted to mzML file format and searched against an Aeromonas hydrophila database downloaded from Uniprot (retrieved 2022 Oct. 24) using MSFragger. The open and closed searches were performed and the parameters were set as those described above. To demonstrate the flexibility of AutoMod, the open and closed searches were performed using MetaMorpheus. For the open search, precursor mass tolerance of 500 Da. and product mass tolerance of 20 ppm were set. No fixed and variable modifications were used. For the closed search, precursor mass tolerance of 20 ppm and product mass tolerance of 20 ppm were set. Carbamidomethylation was specified as the fixed modification. Default values were used for the unmentioned parameters.

### Study Protocol

### PSMs, Peptides, and Proteins Identification

Raw files in mzML file format are processed using search engines with the open search strategy, generating PSMs. The PSMs in the pepXML files are then processed by AutoMod, obtaining recommended modification patterns. Next, the same raw files are searched against with the same databases using search engines with the closed search strategy. The generated PSMs can then be validated using conventional validation tools, such as Percolator, PeptideProphet and ProteinProphet, exporting high confident peptides and proteins.

### The General Workflow of AutoMod Algorithm

AutoMod takes the pepXML files from open searches and user-defined PTMs as input data. To facilitate the usage of AutoMod, a total of 80 PTMs downloaded from UniProt and eight commonly used PTMs are provided in the AutoMod parameter file. Users can modify the PTM lists in the AutoMod parameter file if necessary. With the PTMs (88 PTMs as default) and the number of combination (default value of 3 and no more than 4), such as 1, 2, 3, or 4 combination/combinations, AutoMod first generates all PTM combinations using the depth-first search (DFS) algorithm, where each PTM is considered as a tree node. A total of 117479 PTM combinations were generated in less than one second. Every PTM combination has the total mass sum (called sum_mass in the present disclosure) and a list of PTMs with their localized sites. For each PSM, AutoMod maps its mass shift (also called delta masses, mass difference, or massdiff in the present disclosure, i.e. the mass difference between the theoretical and observed precursor) with the sum_mass of every PTM combination. A massdiff of PSM is matched with a PTM combination if (1) the mass difference between the massdiff and sum_mass is within a user-defined mass tolerance (20 ppm as the default value) and (2) the PTM localized sites are included in the peptide sequence of the PSM. A PTM combination is regarded as matched if it is mapped to at least one PSM. AutoMod groups the matched PTM combinations and exports top N PTM candidates (N is user-defined, 10 as the default value). Users can then apply the exported PTMs for closed searches.

### Results

AutoMod automatically detects possible PTMs from open search results which can then be used for downstream closed searches. The performance of AutoMod is evaluated by using three public proteomic data sets, including one TMT labeled dataset (i.e. ccRCC phosphoproteome dataset) and two label-free datasets (i.e. HEK293 proteome dataset and acetylation-enriched proteome dataset).

### Detection of Variable Modifications from ccRCC Phosphoproteome Data

To demonstrate the performance of AutoMod, a ccRCC phospho-enriched proteomic data with 23 TMT 10-plex (each with 13 fractions) was used for performance evaluation. The data set was labeled with the tandem mass tag (the mass of 229.1629 at lysine (K)) and has phosphorylation at Serine(S), Threonine (T), and Tyrosine (Y).

The open searches were performed individually in each TMT 10-plex using MSFragger and Philosopher via FragPipe, where no modification was specified, and applied AutoMod to detect possible modification candidates from the open search results. The average running time of AutoMod is four seconds per file. Table 1 shows the possible variable modifications AutoMod detected from all 23 TMT 10-plex (all in Table 1) and from each TMT 10-plex (Folder 1 to 23 in Table 1). Comparing the modifications detected by AutoMod and those suggested by FragPipe, AutoMod detected the tandem mass tag of 229.1629 at peptide N-terminus, phosphorylation (79.0663) at Serine(S), and Threonine (T) as those suggested by FragPipe in either individual TMT 10-plex or all 23 TMT 10-plex. In addition, phosphorylation (79.0663) at Aspartate (D), sulfation at Serine(S), and Threonine (T) not suggested by FragPipe were also additionally detected by AutoMod. These results demonstrate that AutoMod can not only detected modifications that are determined based on human experiences, but also discover potential modifications that are outside of our expectation. It was also observed that some modifications (e.g., tandem mass tag at Serine(S), and Threonine (T), and pyrophosphorylation at Serine(S)) were also detected in over 50% of TMT 10-plex, implying the importance of these modifications. Oxidation is often regarded as a common variable modification used for regular protein database searches. Oxidation is not included in the AutoMod-recommended modification candidates because only 153 PSMs have the mass shift of 15.9949.

TABLE 1
The variable modification detected from the 23 TMT 10-plex by AutoMod

| mass | | | | Folder | | | | | | | | | | | | | | | | | | | | | | |
|---|---|---|---|---|---|---|---|---|---|---|---|---|---|---|---|---|---|---|---|---|---|---|---|---|---|---|
| shift | POSN | DEF | all | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |
| 15.9949 | M | V | | | | | | | | | | | | | | | | | | | | | | | | |
| 159.93 | S | | | O | | | | O | | | | | O | | | | | | O | | | O | O | | O | O |
| 229.16 | pep^{N} | | O | O | O | O | O | O | O | O | O | O | O | O | O | O | O | O | O | O | O | O | O | O | O | O |
| | S | | O | O | O | O | O | O | O | O | O | O | O | O | O | O | O | O | O | O | O | O | O | O | O | O |
| | T | | O | O | O | O | O | O | O | O | O | O | O | O | O | O | O | O | O | O | O | | | O | | O |
| 79.95 | S | | O | O | O | O | O | O | O | O | O | O | O | O | O | O | O | O | O | O | O | O | O | O | O | O |
| | T | | O | O | O | O | O | O | O | O | O | O | O | O | O | O | O | O | O | O | O | O | O | O | O | O |
| 79.96 | D | | O | O | O | O | O | O | O | O | O | O | O | O | O | O | O | O | O | O | O | O | O | O | O | O |
| | R | | O | O | O | | | O | O | O | | O | O | | | O | | | O | O | O | O | O | O | O | O |
| | S | V | O | O | O | O | O | O | O | O | O | O | O | O | O | O | O | O | O | O | O | O | O | O | O | O |
| | T | V | O | O | O | O | O | O | O | O | O | O | O | O | O | O | O | O | O | O | O | O | O | O | O | O |
| | Y | V | | | | | | | | | | | | | | | | | | | | | | | | |
| 57.02 | pep^{N} | | | | | | | | | | | | O | | | | | | | | | O | O | O | O | |

Column 4 to 27 shows the modifications detected from each TMT 10-plex and the fourth column shows the modifications detected from all TMT 10-plex. The circle “O” indicates the modifications at the targeted amino acid are detected in the TMT 10-plex. pep^{N} stands for the peptide N terminus. POSN: position; and DEF: default (“V” represents default).

### Identification of Unknown Mass Shifts by AutoMod

Several tools have been proposed for the interpretation of the dark materials, i.e. the unknown mass shifts, in the open search results, such as PTM-Shepherd, which is able to automate characterization of PTMs detected in the open search. The results of PTM-Shepherd were compared with AutoMod. The software comparison was conducted by using the ccRCC phosphoproteome data as the benchmark dataset, and run PTM-Shepherd via FragPipe.

As a result, PTM-Shepherd detected a total of 207 unknown mass shifts where 44 mass shifts matched with known modifications and 163 mass shifts were unannotated. The 207 mass shifts were searched in the AutoMod result and 71% (116 out of 163) unannotated mass shifts were observed to be possibly composed of more than one modification. For example, the mass shift of 309.1292 having 107041 PSMs is unannotated by PTM-Shepherd but is annotated by AutoMod as the combination of phosphorylation and TMT6plex (Table 2). AutoMod is also capable of detecting the mass shifts matching with single modification (e.g., the mass shift of 79.9663 is corresponding to phosphorylation). These results suggest that AutoMod enables users to explore the possible modification combinations, providing a more general view of interpreting the unknown mass shifts.

TABLE 2
The top six unknown mass shifts interpreted by PTM-Shepherd and AutoMod

| Mass | Modification | | Number of PSMs | |
|---|---|---|---|---|
| Shift | PTM-Shepherd | AutoMod | PTM-Shepherd | AutoMod |
| 309.1292 | Unannotated | Phosphorylation and | 107041 | 70087 |
| | mass-shift | TMT6plex | | |
| 229.1629 | TMT6plex | TMT6plex | 27177 | 31203 |
| 389.0901 | EZ-Link | Pyrophospho and | 16962 | 15764 |
| | Sulfo-NHS-SS-Biotin | TMT6plex | | |
| 79.9663 | Phosphorylation | Phosphorylation | 6562 | 4186 |
| 366.1518 | Unannotated | Carbamidomethyl, | 8747 | 4097 |
| | mass-shift | Phosphorylation, | | |
| | | and TMT6plex | | |
| 325.1246 | Unannotated | Oxidation, | 6597 | 3699 |
| | mass-shift | Phosphorylation, | | |
| | | and TMT6plex | | |

### Improvement of Protein Identification via Customized Modifications

To validate the modifications detected by AutoMod, four closed searches, including (1) a closed search using no variable modifications (none), (2) a closed search using default variable modifications suggested by FragPipe (default), (3) a closed search using the modifications customized for each TMT 10-plex (customized) and (4) a closed search using the modifications detected from all TMT 10-plex by AutoMod (all) were then performed, and their results were compared. Other search parameters were the same and described in the METHODOLOGY. The terms “none,” “default,” “customized,” and “all” are used in the following paragraphs to represent the results of the four closed search.

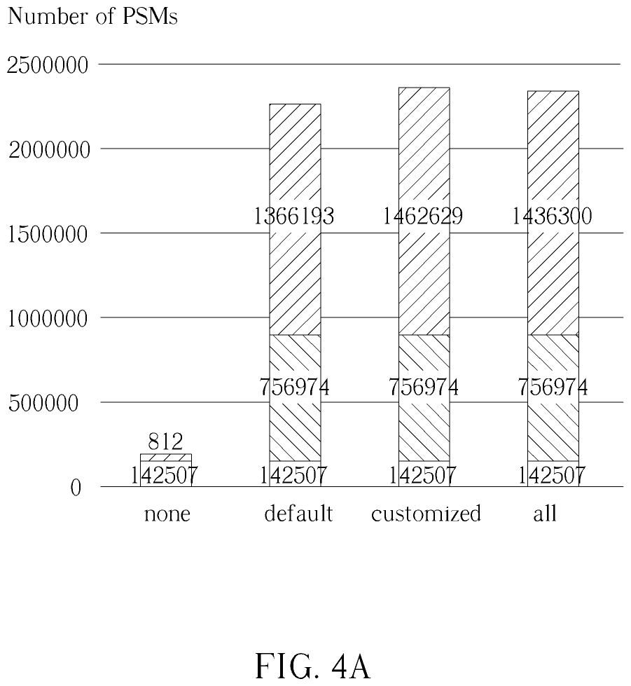

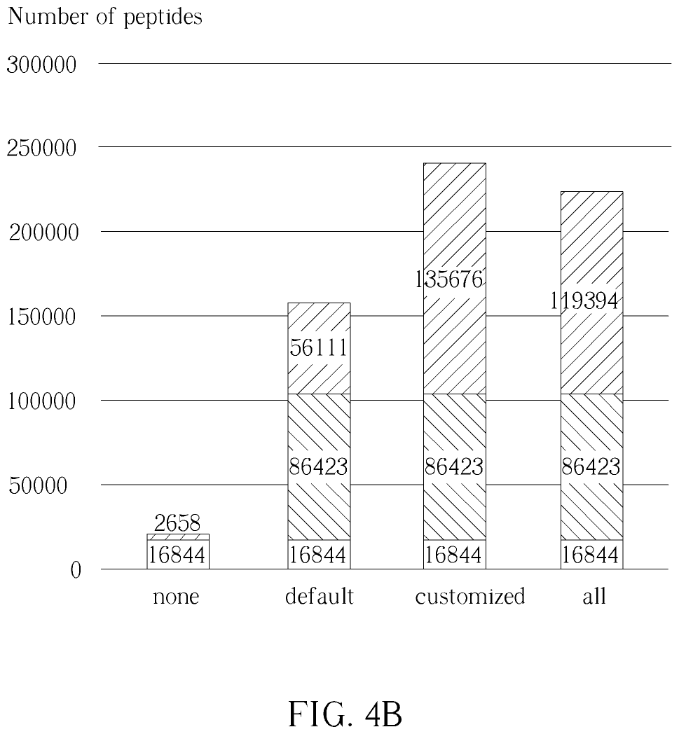

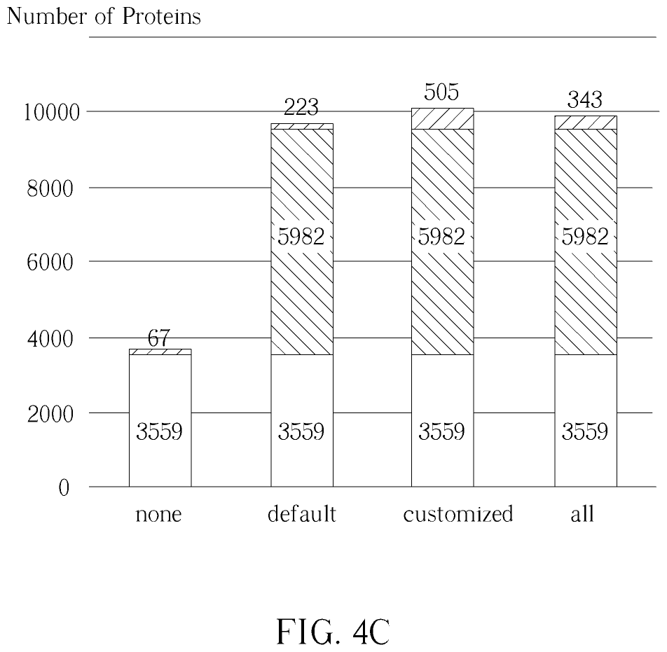

FIGS. 4A, 4B, and 4C show the number of identified PSMs, peptides, and proteins using the four modification settings. As expected, searching with no variable modification results the smallest number of identified PSMs, peptides, and proteins. Meanwhile, using customized search setting results the most identified peptides and proteins. Further comparing the modifications identified by the default, customized and all parameter settings, it was observed that sulfation at serine(S), and threonine (T), and TMT6plex at serine(S), and threonine (T) were identified using the parameter setting of “customized” and “all,” and pyrophospho at serine(S) were additionally identified using the customized parameter setting, demonstrating the ability of AutoMod in providing proper modification candidates (as shown in Table 3).

TABLE 3
The modifications identified by the parameter
setting of default, customized, and all

| modifications | mass | amino acid | default | Customized | all |
|---|---|---|---|---|---|
| Oxidation | 15.9949 | M | 272106 | 0 | 0 |
| Carbamidomethyl | 57.0215 | C | 392526 | 435550 | 430727 |
| Sulfoserine | 79.9568 | S | 0 | 6205 | 1209 |
| | | T | 0 | 1985 | 491 |
| | | D | 0 | 288328 | 299542 |
| | | R | 0 | 73929 | 117971 |
| Phosporylation | 79.9663 | S | 2166894 | 1781482 | 1859719 |
| | | T | 380756 | 333585 | 341838 |
| | | Y | 69932 | 0 | 0 |
| Pyrophospho | 159.9327 | S | 0 | 18791 | 0 |
| | | N-term | 1706548 | 1735555 | 1716980 |
| TMT6plex | 229.1629 | K | 2265674 | 2310721 | 2302968 |
| | | S | 0 | 23988 | 8221 |
| | | T | 0 | 3568 | 1137 |

### Demonstration of the AutoMod Robustness

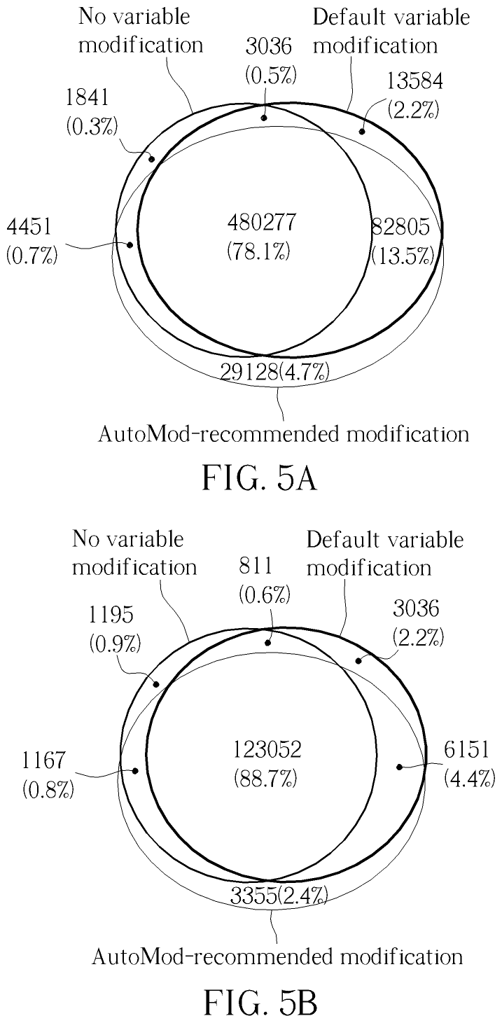

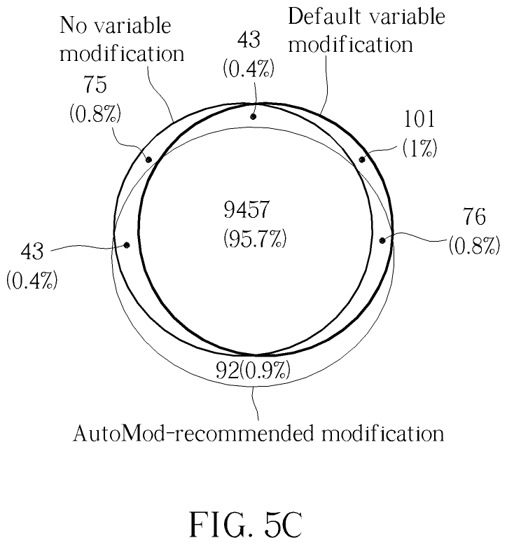

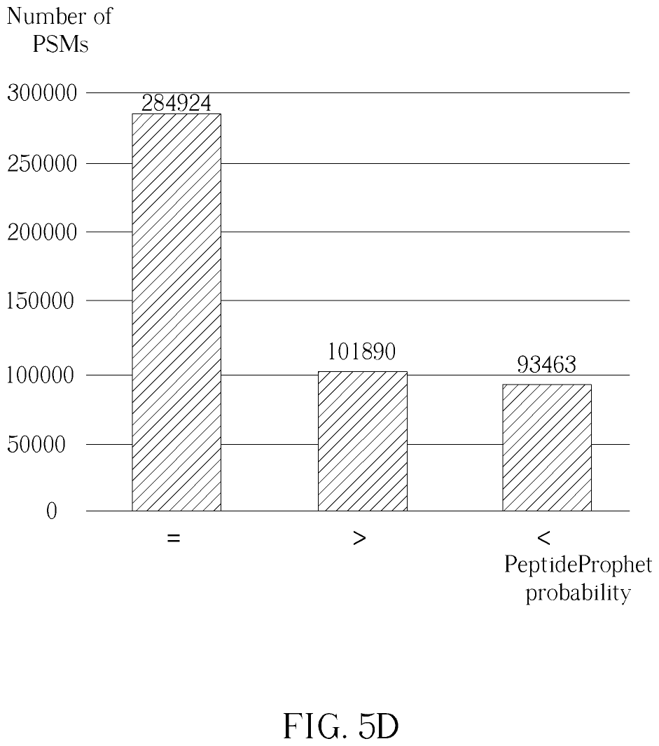

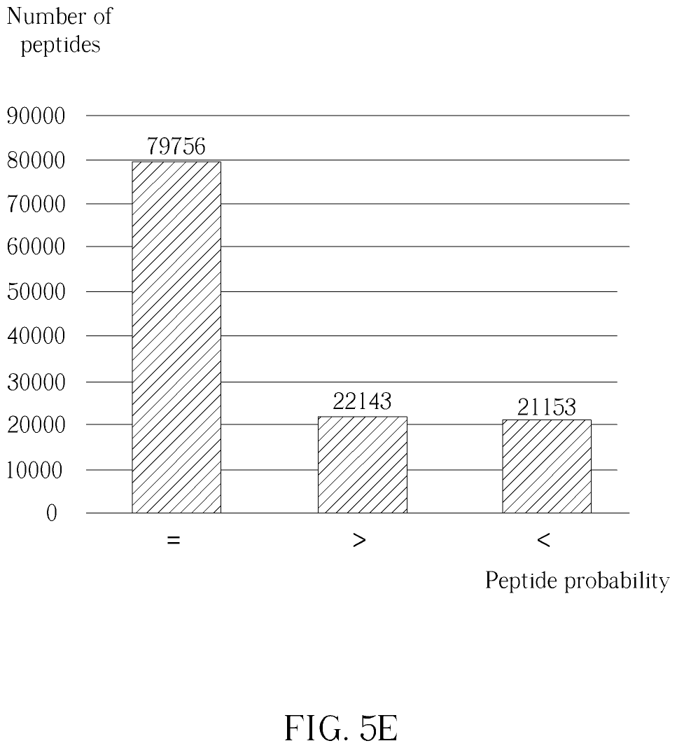

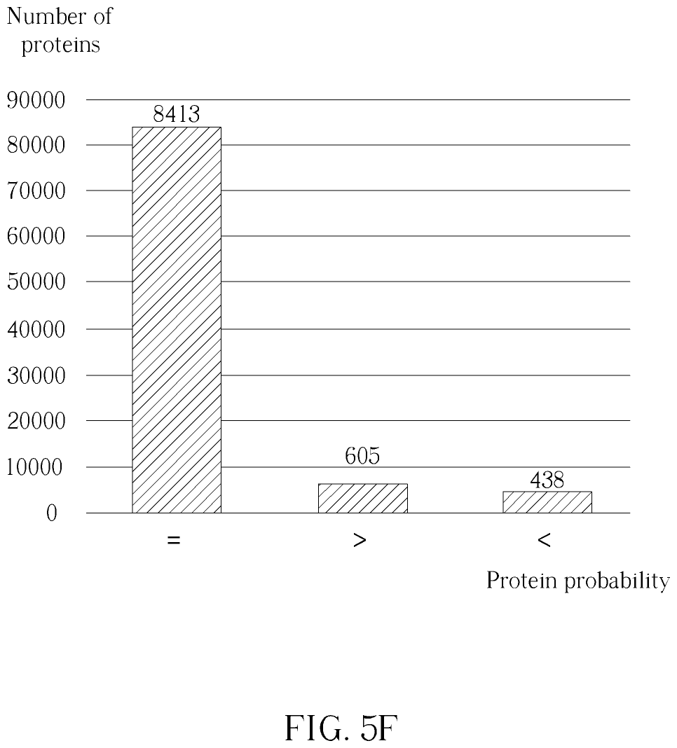

To demonstrate the robustness of AutoMod, a label-free HEK293 proteomic data set was used for performance evaluation. An open search using MSFragger and Philosopher via FragPipe without specifying any variable modifications was performed. The open search result was then processed by AutoMod and top 10 modification patterns were selected as AutoMod-recommended variable modifications. Next, three closed searches were conducted using MSFragger and Philosopher via FragPipe. Three variable modification settings, including no variable modification, the default variable modifications, and AutoMod-recommended modifications, were specified in the three closed searches, respectively. The three closed searches as no modification search, default search, and AutoMod-recommended search were used in the following paragraphs. As a result, 489605 PSMs, 126225 peptides, and 9618 proteins were identified using no modification search, 579702 PSMs, 133050 peptides, and 9677 proteins were identified using the default search, and 596661 PSMs, 133725 peptides, and 9668 proteins were identified using the AutoMod-recommended search (as shown in FIGS. 5A, 5B, and 5C). Comparing the three search results, 480277 (78.1%) PSMs, 123052 (88.7%) peptides, and 9457 (95.7%) proteins were commonly identified by the three searches, and 1841 (0.3%), 13584 (2.2%), and 29128 (4.7%) PSMs, 1195 (0.9%), 3036% (2.2%), and 3355 (2.4%) peptides, 75 (0.8%), 101 (1%), and 92 (0.9%) proteins were additionally identified by the no modification, default, and the AutoMod-recommended searches, respectively (FIGS. 5A, 5B, and 5C).

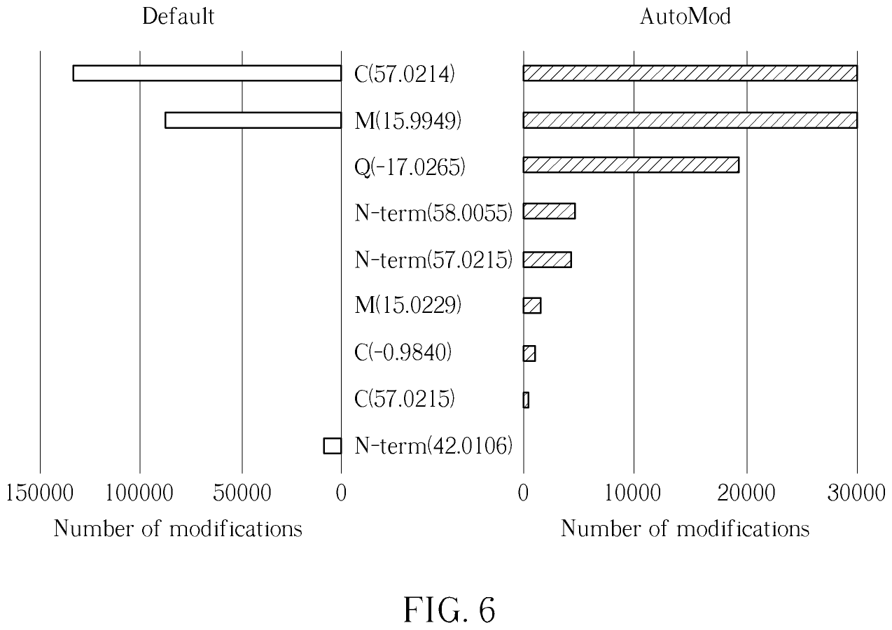

Furthermore, the PeptideProphet probability, peptide probability and protein probability of commonly identified PSMs, peptides, and proteins were compared to investigate whether the identification accuracy increases using the AutoMod-recommended search. As a result, comparing to the default search, the probabilities of 101890 PSMs, 22143 peptides, and 605 proteins increase using the AutoMod-recommended search (FIGS. 5D, 5E, and 5F). The modifications identified using the default and AutoMod searches were also compared. As shown in FIG. 6, more modifications were identified using the AutoMod-recommended modifications.

These results demonstrate that the AutoMod-recommended search not only identifies more PSMs and peptides than the no modification and default searches, but also has higher PSM, peptides, and protein probabilities. More importantly, AutoMod enables the discovery of potential modifications which are later identified as valuable modifications by downstream closed searches.

### Demonstration of the AutoMod Flexibility

Many database search engines have implemented the open search strategy as an option. To demonstrate that AutoMod can be adapted with different search engines, MetaMorpheus is used to process a public label-free acetylation-enriched proteomic dataset. An open search was first performed using MetaMorpheus, and AutoMod was then used to detect possible modifications from the search result. Table 4 shows the top three modification candidates detected from the open search result by AutoMod, where acetylation is successfully detected as the most possible modification.

TABLE 4
Top 3 modification candidates detected by AutoMod

| | Possible | Number of | | |
|---|---|---|---|---|
| mass | sites | matches | Confidence | Support |
| 42.0106 | 42.0106@K | 3300 | 99.90917348 | 3.178394622 |
| 42.0226 | −1.0316@K; | 2372 | 71.55354449 | 2.284591528 |
| | 43.0542@A | | | |
| 42.0218 | −0.984@A; | 2371 | 71.56655599 | 2.283628378 |
| | 43.0058@K | | | |

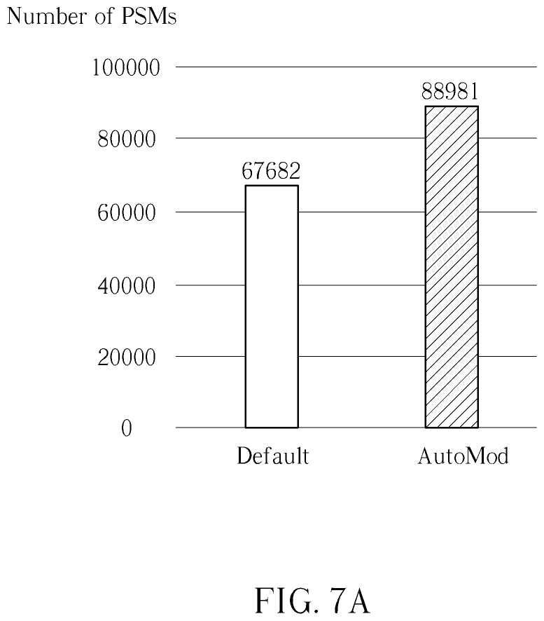

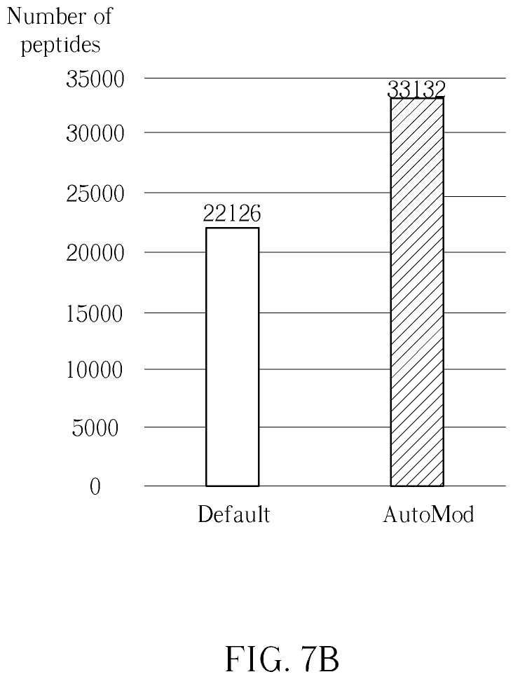

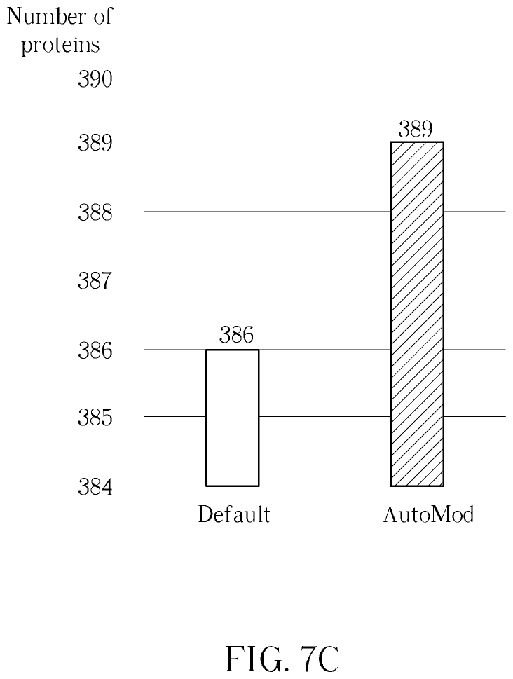

Next, two closed searches were performed using MetaMorpheus, including one with the modification setting described in the publication of acetylation-enriched proteomic dataset (Zhang, L., et al. Mol Cell Proteomics 2022, 21 (9), 100248) and another with AutoMod-recommended modifications. The detailed processing is described in the Methodology section. As shown in FIGS. 7A, 7B, and 7C, more PSMs, peptides, and proteins were identified using AutoMod-recommended modification search. The result demonstrates the flexibility of AutoMod being able to be adapted to different search engines.

Based on the description above, discovery of PTMs is an important task in the field of proteomics. An efficient software tool, AutoMod, is implemented for PTMs detection. According to the evaluation using three proteomics datasets described herein, it is demonstrated that AutoMod can automatically detect major PTMs from proteomics data, and more PSMs, peptides, and proteins are identified using AutoMod-recommended modifications. It is also demonstrated the flexibility and robustness of AutoMod that it can be adapted with different search engines, such as MSFragger and Metamorpheus, and the performance is stable and outperformed.

With AutoMod, users can discover unexpected PTMs from proteomics datasets. Meanwhile, the evaluation using the ccRCC phospho-enriched dataset has also shown that the modifications might be varied among different batches, and it is important to have a software tool that can automatically uncover possible PTMs from experiments, achieving the customized modification settings.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for automatic detection of post-translational modifications of a biological compound, comprising:
acquiring a mass shift of a compound-spectrum match between the biological compound and a spectrum;
generating post-translational modification combinations and masses thereof by processing user-defined post-translational modifications using a search algorithm;
matching the mass shift and a mass of each of the post-translational modification combinations; and
automatically detecting the post-translational modifications of the biological compound based on the matching.

2. The method of claim 1, further comprising using a position obtained from the compound-spectrum match to validate the correctness of matched post-translational modification combinations.

3. The method of claim 2, wherein the post-translational modification combinations of the biological compound is detected by the following conditions: 1) a mass difference between the mass shift of the compound-spectrum match and the mass of each of the post-translational modification combinations is within a default value, and 2) locations of post-translational modifications in the post-translational modification combinations are included in a sequence of the compound-spectrum match.

4. The method of claim 3, further comprising exporting matched post-translational modification combinations mapping to the compound-spectrum match.

5. The method of claim 1, wherein the search algorithm is a depth-first search algorithm.

6. The method of claim 5, wherein each of the user-defined post-translational modifications is a tree node of the depth-first search.

7. The method of claim 1, wherein the biological compound is a protein, and the compound-spectrum match is a peptide-spectrum match.

8. The method of claim 1, further comprising acquiring the compound-spectrum match and a mass thereof from an open search strategy.

9. The method of claim 8, further comprising exporting the post-translational modification combinations to a closed search strategy for identification and validation.

10. A system for automatic detection of post-translational modifications of a biological compound, comprising:
a memory for storing user-defined post-translational modifications and a mass shift of a compound-spectrum match between the biological compound and a spectrum; and
a processor for:
generating post-translational modification combinations and masses thereof by processing the user-defined post-translational modifications using a search algorithm;
matching the mass shift and a mass of each of the post-translational modification combinations; and
automatically detecting the post-translational modification combinations of the biological compound based on the matching.

11. The system of claim 10, wherein the processor is configured for using a position obtained from the compound-spectrum match to validate the correctness of matched post-translational modification combinations, and wherein the post-translational modification combinations of the biological compound is detected by the following conditions: 1) a mass difference between the mass shift of the compound-spectrum match and the mass of each of the post-translational modification combinations is within a default value, and 2) locations of post-translational modifications in the post-translational modification combinations are included in a sequence of the compound-spectrum match.

12. The system of claim 10, wherein the search algorithm is a depth-first search algorithm, and each of the user-defined post-translational modifications is a tree node of the depth-first search.

13. The system of claim 10, wherein the processor is configured for:
acquiring the compound-spectrum match and a mass thereof from an open search strategy; and
exporting the post-translational modification combinations to a closed search strategy for identification and validation.

14. A computer readable medium with an executable instruction stored thereon to perform the method for automatic detection of post-translational modifications of a biological compound, wherein the method comprises:
acquiring a mass shift of a compound-spectrum match between the biological compound and a spectrum;
generating post-translational modification combinations and masses thereof by processing user-defined post-translational modifications using a search algorithm;
matching the mass shift and a mass of each of the post-translational modification combinations; and
automatically detecting the post-translational modifications of the biological compound based on the matching.

15. The computer readable medium of claim 14, wherein the method further comprises using a position obtained from the compound-spectrum match to validate the correctness of matched post-translational modification combinations.

16. The computer readable medium of claim 15, wherein the post-translational modification combinations of the biological compound is detected by the following conditions: 1) a mass difference between the mass shift of the compound-spectrum match and the mass of each of the post-translational modification combinations is within a default value, and 2) locations of post-translational modifications in the post-translational modification combinations are included in a sequence of the compound-spectrum match.

17. The computer readable medium of claim 14, wherein the search algorithm is a depth-first search algorithm.

18. The computer readable medium of claim 17, wherein each of the user-defined post-translational modifications is a tree node of the depth-first search.

19. The computer readable medium of claim 14, wherein the method further comprises acquiring the compound-spectrum match and a mass thereof from an open search strategy.

20. The computer readable medium of claim 19, wherein exporting the post-translational modification combinations to a closed search strategy for identification and validation.
